(19) United States
(12) Patent Application Publication
(10) Pub. No.: US 2025/0208277 A1
(43) Pub. Date: Jun. 26, 2025

(54) SYSTEMS AND METHODS FOR COLOR MAPPING FOR CONTRAST ENHANCED ULTRASOUND PARAMETRIC IMAGING

(71) Applicant: KONINKLIJKE PHILIPS N.V., EINDHOVEN (NL)

(72) Inventors: Shiying Wang, Melrose, MA (US); Thanasis Loupas, Kirkland, WA (US); William Tao Shi, Wakefield, MA (US); Mingxin Zheng, Cambridge, MA (US)

(21) Appl. No.: 18/851,971

(22) PCT Filed: Mar. 29, 2023
(86) PCT No.: PCT/EP2023/058072

§ 371 Date: Sep. 27, 2024

Related U.S. Application Data
(60) Provisional application No. 63/326,033, filed on Mar. 31, 2022.

(51) Int. Cl. G01S 7/52; G01S 7/62; G01S 15/89

(52) U.S. Cl. CPC G01S 7/52071; G01S 7/52026; G01S 7/6263; G01S 15/8988

(57) ABSTRACT

A color map may be used to assign color hue or brightness values to pixels of images based on intensity values of ultrasound signals. The color map may be dynamic rather than fixed with respect to time or another parameter. In some examples, a range of color hue or brightness values assigned to intensity values may dynamically change with image number of a sequence. In some examples, the color map may change for individual images of the sequence. In some examples, the color map may change over time. In some examples, the color hue and/or brightness distribution of the color map is linear with time. In some examples the color hue and/or brightness distribution of the color map is adapted based, at least in part, on properties of the images.

### TECHNICAL FIELD

This application relates to contrast-enhanced ultrasound imaging. More specifically, this application relates to mappings, such as color mappings, of parametric images derived from contrast-enhanced ultrasound image sequences.

### BACKGROUND

Contrast-enhanced ultrasound (CEUS) is a technique that has been increasingly used in the field of clinical diagnosis. The CEUS technique is based on the administration of an ultrasound contrast agent (UCA) to a patient, for example, phospholipid-stabilized gas-filled microbubbles. The UCA acts as an ultrasound reflector and can be detected by measuring echo signals that are returned in response to ultrasound waves. Since the UCA flows through the vasculature of the patient, the detection of the UCA provides information about blood perfusion in a region of interest (ROI) in the patient.

In imaging by the CEUS technique, an image sequence that corresponds to a time evolution of the UCA in the ROI during a blood perfusion process or other physiological process is generated (e.g., temporal image sequence, sequence of temporally spaced images), in which a value of a pixel in an image represents an intensity of measured echo signals from the UCA at a time the image was acquired. Thus, the generated image sequence provides information of the blood perfusion in the ROI in the patient. Further examination of the measured echo signals provides parameters, such as a time-of-arrival (TOA), a time-to-peak, a wash-in rate, and a wash-out rate, at each point in the ROI. The parameters may be shown in color-coded parametric images.

Parametric images are generated using a mapping between the value of a parameter at each point in the ROI and a color hue value and/or a brightness value at each pixel in an image. While a color hue value and/or a brightness value may be mapped to a parameter value, all mappings will be collectively referred to as color mappings. A color-coded parametric image generated using a color mapping may be displayed independently or overlaid on the corresponding images of the image sequence to provide a sequence of parametric images. This may allow a user to observe changes in a parameter across the sequence (e.g., over time). For example, the parametric images based, at least in part, on the TOA parameter, may allow a user to observe when the UCA arrived at different portions of the ROI.

### SUMMARY

Systems and methods are described for forming a time sequence of parametric images which provide visual information about time parameters related to a region of interest (ROI) in a subject based on contrast-enhanced ultrasound (CEUS) imaging. In the embodiments described herein, a color mapping between a time parameter related to the ROI to a color hue and/or brightness value of a pixel in an image is adjusted for each image. Specifically, the color map is dynamically changed based on a time point within the entire time sequence and/or the time parameters determined at all or some of the time points within the entire time sequence. Due to the dynamic adjustment of the color mapping, resulting color-coded images include larger variation in color hue and/or brightness and thus provide increased visual distinguishability from one pixel to another.

In accordance with at least one example disclosed herein, an ultrasound imaging system may include an input configured to receive ultrasound echo signals from a region of interest (ROI) to generate a sequence comprising a plurality of temporally spaced color-coded parametric images of the ROI and a processor configured to calculate a parameter for each pixel in each image in the sequence; generate a color map of the parameter for each image in the sequence, wherein the color map includes values of a color hue, a brightness, or a combination thereof to be assigned to each pixel of the image, based at least in part on a range of the calculated parameter to be color coded, wherein a range of the values of the color map is determined based, at least in part, on a temporal location of the corresponding image within the sequence; and generate the plurality of temporally spaced color-coded parametric images based on the color map of the parameter for each image in the sequence.

In some examples, the color map of the parameter varies linearly as a function of time. In some examples, the color map of the parameter varies adaptively based on a change rate of a spatially averaged value of the parameter. In some examples, the color map of the parameter varies adaptively based on an occupation ratio. In some examples, the occupation ratio is calculated based, at least in part, on a ratio of pixels including a contrast agent and a total number of pixels in an image of the plurality of temporally spaced images. In some examples, the color map of the parameter varies adaptively based on a percentile of the parameter.

In some examples, the ultrasound imaging system may further include a display configured to display the plurality of temporally spaced color-coded parametric images.

In some examples, the input may include an ultrasound probe.

In accordance with at least one example disclosed herein, a method of forming a color-coded parametric image sequence may include calculating a parameter for each pixel in each image in the sequence; generating a color map of the parameter for each image in the sequence, wherein the color map includes values of a color hue, a brightness, or a combination thereof to be assigned to each pixel of the image, based at least in part on a range of the calculated parameter to be color coded, wherein a range of the values of the color map is determined based, at least in part, on a temporal location of the corresponding image within the sequence; and generating the plurality of temporally spaced color-coded parametric images based on the color map of the parameter for each image in the sequence.

In some examples, the parameter comprises a time of arrival (TOA), a time-to-peak, a wash-in rate, a wash-out rate, a concentration, a flow rate, a perfusion rate, or a combination thereof.

In some examples, the method further includes performing contrast enhanced ultrasound imaging to acquire the plurality of temporally spaced images of the sequence.

In some examples, the color map of the parameter varies adaptively based on an occupation ratio. In some examples, the occupation ratio is calculated based, at least in part, on a ratio of pixels including a contrast agent and a total number of pixels in an image of the plurality of temporally spaced images. In some examples, the color map of the parameter varies linearly as a function of time. In some examples, the color map of the parameter varies adaptively based on a change rate of a spatially averaged value of the parameter. In some examples, the color map of the parameter varies adaptively based on a percentile of the parameter.

In accordance with at least one example disclosed herein, a non-transitory computer readable medium may include instructions that when executed cause an ultrasound imaging system to calculate a parameter for each pixel in each image in the sequence; generate a color map of the parameter for each image in the sequence, wherein the color map includes values of a color hue, a brightness, or a combination thereof to be assigned to each pixel of the image, based at least in part on a range of the calculated parameter to be color coded, wherein a range of the values of the color map is determined based, at least in part, on a temporal location of the corresponding image within the sequence; and generate the plurality of temporally spaced color-coded parametric images based on the color map of the parameter for each image in the sequence.

In some examples, the non-transitory computer readable medium may further include instructions that when executed cause the ultrasound imaging system to select the parameter to calculate from a plurality of parameters based, at least in part, on a user input received from a user interface. In some examples, the plurality of parameters comprises a time of arrival (TOA), a time-to-peak, a wash-in rate, a wash-out rate, a concentration, a flow rate, a perfusion rate, or a combination thereof.

In some examples, the color map of the parameter varies linearly as a function of time or varies adaptively based on an occupation ratio or a change rate of a spatially averaged value of the parameter.

In some example, non-transitory computer readable medium may further include instructions that when executed cause the ultrasound imaging system to display the plurality of temporally spaced color-coded parametric images on a display.

### BRIEF DESCRIPTION OF THE DRAWINGS

FIG. 1 is a block diagram of an ultrasound imaging system arranged in accordance with some examples of the present disclosure.

FIG. 2 is a block diagram illustrating an example processor in accordance with some examples of the present disclosure.

FIGS. 3A, 3B, 3C, and 3D are example TOA parametric images of image sequences of a kidney generated by various CEUS techniques, including those in accordance with some examples of the present disclosure.

FIG. 4 is a flow chart of a method according to an embodiment of the present disclosure.

### DESCRIPTION

The following description of certain exemplary examples is merely exemplary in nature and is in no way intended to limit the invention or its applications or uses. In the following detailed description of examples of the present systems and methods, reference is made to the accompanying drawings which form a part hereof, and in which are shown by way of illustration specific examples in which the described systems and methods may be practiced. These examples are described in sufficient detail to enable those skilled in the art to practice the presently disclosed systems and methods, and it is to be understood that other examples may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

As previously mentioned, parametric images may be generated for UCA image sequences by mapping color hue values and/or brightness values of pixels to values of one or more parameters (e.g., TOA, time-to-peak). The color mapping may be based on a fixed (e.g., predetermined) color mapping (predetermined. e.g., a table that defines the hue/luminance or Red/Blue/Green values to be used to color code each value of the parameter of interest) for all images in an image sequence irrespective of a length of the image sequence (e.g., a number of images in the sequence and/or a period of time over which the image sequence was acquired). However, the fixed color mapping may result in loss of visually perceived information in some instances. For example, a TOA parametric image in which early arrivals of a UCA are represented by pink and red pixels, and late arrivals of the UCA are represented by blue pixels, may be dominated in pink and red with no visible yellow, green, or blue, when a TOA of most UCA is in an early phase of an image sequence. This reduces contrast of the TOA parametric images, leading to visual indistinguishability from one pixel to another in the TOA parametric images. This may make it difficult for a user to observe which areas of the ROI received the UCA first when arrival times are close to each other.

According to examples of the present disclosure, mapping of a parameter value to a color hue value and/or a brightness value may vary to generate a parametric image for each UCA image in an image sequence. In some examples, the color map is dynamically changed based on a time point within a time sequence and/or the values of parameters determined at all or some of the time points within the time sequence. Due, at least in part, to the dynamic adjustment of the color mapping, resulting parametric images may include larger variation in colors and/or intensities. This may provide increased visual distinguishability from one pixel to another.

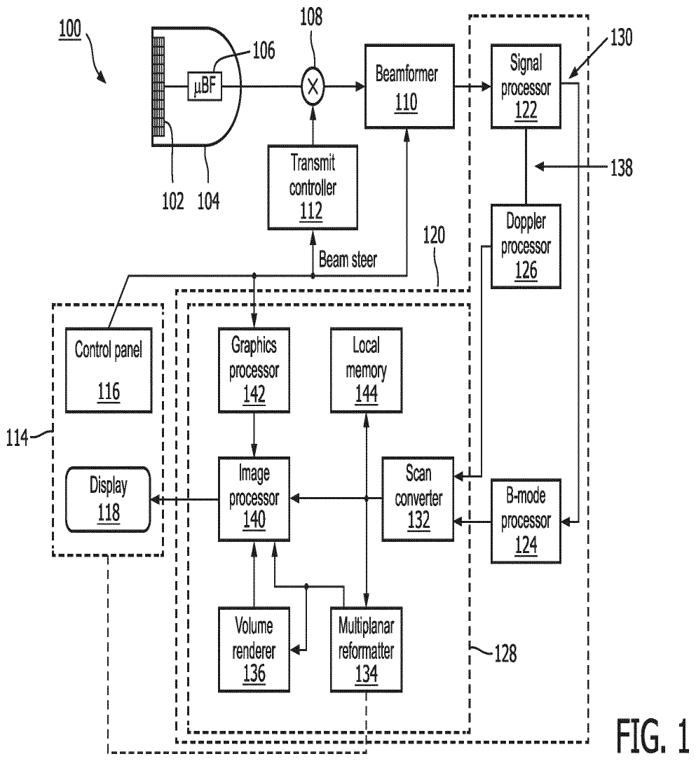

FIG. 1 shows a block diagram of an ultrasound imaging system 100 constructed in accordance with the principles of the present disclosure. An ultrasound imaging system 100 according to the present disclosure may include an input of ultrasound echo signals. An example input may include a transducer array 102, which may further be included in an ultrasound probe 104, for example, an external probe or an internal probe such as an intravascular ultrasound (IVUS) catheter probe. In other examples, the transducer array 102 may be in the form of a flexible array configured to be conformally applied to a surface of subject to be imaged (e.g., patient). The transducer array 102 is configured to transmit ultrasound signals (e.g., beams, waves) and receive echoes (e.g., received ultrasound signals) responsive to the transmitted ultrasound signals. A variety of transducer arrays may be used, e.g., linear arrays, curved arrays, or phased arrays. The transducer array 102 can include, for example, a two dimensional array (as shown) of transducer elements capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction.

In some examples, the transducer array 102 may be coupled to a microbeamformer 106, which may be located in the ultrasound probe 104, and which may control the transmission and reception of signals by the transducer elements in the array 102. In some examples, the microbeamformer 106 may control the transmission and reception of signals by active elements in the array 102 (e.g., an active subset of elements of the array that define the active aperture at any given time).

In some examples, the microbeamformer 106 may be coupled, e.g., by a probe cable or wirelessly, to a transmit/receive (T/R) switch 108, which switches between transmission and reception and protects a main beamformer 110 from high energy transmit signals. In some examples, for example in portable ultrasound systems, the T/R switch 108 and other elements in the system can be included in the ultrasound probe 104 rather than in an ultrasound system base, which may house the image processing electronics. An ultrasound system base typically includes software and hardware components including circuitry for signal processing and image data generation as well as executable instructions for providing a user interface.

The transmission of ultrasonic signals from the transducer array 102 under control of the microbeamformer 106 is directed by a transmit controller 112, which may be coupled to the T/R switch 108 and the main beamformer 110. The transmit controller 112 may control the direction in which beams are steered. Beams may be steered straight ahead from (orthogonal to) the transducer array 102, or at different angles for a wider field of view. The transmit controller 112 may also be coupled to a user interface 114 and receive input from the user's operation of a user control. The user interface 114 may include one or more input devices such as a control panel 116, which may include one or more mechanical controls (e.g., buttons, encoders, etc.), touch sensitive controls (e.g., a trackpad, a touchscreen, or the like), and/or other known input devices. The user interface 114 may further include a display 118.

In some examples, the partially beamformed signals produced by the microbeamformer 106 may be coupled to the main beamformer 110 where partially beamformed signals from individual patches of transducer elements may be combined into a fully beamformed signal. In some examples, microbeamformer 106 is omitted, and the transducer array 102 is under the control of the main beamformer 110 and the main beamformer 110 performs all beamforming of signals. In examples with and without the microbeamformer 106, the beamformed signals of the main beamformer 110 are coupled to processing circuitry 120, which may include one or more processors (e.g., a signal processor 122, a B-mode processor 124, a Doppler processor 126, and one or more image generation and processing components 128) configured to produce an ultrasound image from the beamformed signals (i.e., beamformed RF data).

The signal processor 122 may be configured to process the received beamformed RF data in various ways, such as bandpass filtering, decimation, I and Q component separation, and harmonic signal separation. The signal processor 122 may also perform additional signal enhancement such as speckle reduction, signal compounding, and electronic noise elimination. The processed signals (also referred to as I and Q components or IQ signals) may be coupled to additional downstream signal processing circuits for image generation. The IQ signals may be coupled to a plurality of signal paths within the system, each of which may be associated with a specific arrangement of signal processing components suitable for generating different types of image data (e.g., B-mode image data, Doppler image data). For example, the system may include a B-mode signal path 130 which couples the signals from the signal processor 122 to the B-mode processor 124 for producing B-mode image data.

The B-mode processor 124 can employ amplitude detection for the imaging of structures in the body. According to principles of the present disclosure, the B-mode processor 124 may generate signals for tissue images and/or contrast images. The signals produced by the B-mode processor 124 may be coupled to a scan converter 132 and/or a multiplanar reformatter 134. The scan converter 132 may be configured to arrange the echo signals from the spatial relationship in which they were received to a desired image format. For instance, the scan converter 132 may arrange the echo signal into a two dimensional (2D) sector-shaped format, or a pyramidal or otherwise shaped three dimensional (3D) format. In another example of the present disclosure, the scan converter 132 may arrange the echo signals into side-by-side contrast enhanced and tissue images.

The multiplanar reformatter 134 can convert echoes which are received from points in a common plane in a volumetric region of the body into an ultrasonic image (e.g., a B-mode image) of that plane, for example as described in U.S. Pat. No. 6,443,896 (Detmer). The scan converter 132 and the multiplanar reformatter 134 may be implemented as one or more processors in some examples.

A volume renderer 136 may generate an image (also referred to as a projection, render, or rendering) of the 3D dataset as viewed from a given reference point. The volume renderer 136 may be implemented as one or more processors in some examples. The volume renderer 136 may generate a render, such as a positive render or a negative render, by any known or future known technique such as surface rendering and maximum intensity rendering.

In some examples, the system may include a Doppler signal path 138 which couples the output from the signal processor 122 to the Doppler processor 126. The Doppler processor 126 may be configured to estimate the Doppler shift and generate Doppler image data. The Doppler image data may include color data which is then overlaid with B-mode (i.e. grayscale) image data for display. The Doppler processor 126 may be configured to filter out unwanted signals (i.e., noise or clutter associated with non-moving tissue), for example using a wall filter. The Doppler processor 126 may be further configured to estimate velocity and power in accordance with known techniques. For example, the Doppler processor may include a Doppler estimator such as an auto-correlator, in which velocity (Doppler frequency) estimation is based on the argument of the lag-one autocorrelation function and Doppler power estimation is based on the magnitude of the lag-zero autocorrelation function. Motion can also be estimated by known phase-domain (for example, parametric frequency estimators such as MUSIC, ESPRIT, etc.) or time-domain (for example, cross-correlation) signal processing techniques. Other estimators related to the temporal or spatial distributions of velocity such as estimators of acceleration or temporal and/or spatial velocity derivatives can be used instead of or in addition to velocity estimators. In some examples, the velocity and power estimates may undergo further threshold detection to further reduce noise, as well as segmentation and post-processing such as filling and smoothing. The velocity and power estimates may then be mapped to a desired range of display colors in accordance with a color map. The color data, also referred to as Doppler image data, may then be coupled to the scan converter 132, where the Doppler image data may be converted to the desired image format and overlaid on the B-mode image of the tissue structure to form a color Doppler or a power Doppler image. For example, Doppler image data may be overlaid on a B-mode image of the tissue structure.

Output (e.g., B-mode images, Doppler images) from the scan converter 132, the multiplanar reformatter 134, and/or the volume renderer 136 may be coupled to an image processor 140 for further enhancement, buffering and temporary storage before being displayed on the display 118.

The image processor 140 may assign a color hue value and/or a brightness value to pixels of each image in a sequence of images based on a two-dimensional (2D) color map to generate a parametric image. The values may be provided by the image processor 140 to the display 118. The parametric image may be displayed on display 118 separately or overlaid on a B-mode image in some examples. The values may define a color hue value and/or a brightness value with which the pixel appears on the display 118. The 2D color map may define a relationship between an intensity of an ultrasound signal at a location corresponding to the pixel and the color hue value and/or brightness value of the pixel for a given point in time. The color hue value and/or brightness value that corresponds to an intensity of the ultrasound signal may vary over time (e.g., across images of the sequence acquired at different points in time). Thus, one dimension of the 2D color map may be intensity and another dimension for the 2D color map may be time.

The 2D color map may be generated by the image processor 140. In some embodiments, how the color hue value and/or brightness value changes with intensity over time may be predefined. For example, a rate at which the color hue value and/or brightness value changes for a given intensity level may be predefined. In some embodiments, the rate of change may be based at least in part, on a type of organ being imaged (e.g., liver, thyroid), a type of contrast agent used, image acquisition settings (e.g., gain, transmit frequency), and/or a type of parameter being studied (e.g., time-of-arrival, clearance).

According to embodiments of the present disclosure, the 2D color map may be dynamic. That is, the 2D color map may vary based on one or more factors. In some embodiments, how the color hue value and/or brightness value changes with intensity over time, such as the rate of change, may vary based on analysis of the images of the sequence either in real time or in post-processing. For example, the image processor 140 may analyze an image in the sequence to determine the intensities of the ultrasound signals for all of the pixels in the image and may adjust the intensity scale of the 2D color map for one or more additional images in the sequence so that no more than a threshold value of pixels are at a peak value for the color hue value and/or brightness value in the additional image(s). The threshold value may be a percentage in some examples (e.g., 0.1%, 1%, 5%). In some examples, rate of change of the color hue value and/or brightness value for a given intensity may be based, at least in part, on a parameter calculated by the image processor 140. For example, one or more images of the sequence may be analyzed to determine a time-of arrival (TOA), a time-to-peak, a wash-out rate, and/or other desired parameter of a contrast agent (e.g., concentration, flow rate, perfusion rate). Based on the calculated parameter, the 2D color map may be determined for individual images of the sequence and color hue value and/or brightness value of the pixels may then be assigned.

Although reference is made to pixels, it is understood that the principles of the present disclosure may also be applied to voxels of three dimensional images.

In some embodiments, whether the 2D color map corresponds to a change in brightness corresponding to an intensity over time, a change in color hue corresponding to an intensity over time, or a combination thereof, may be determined by a user, for example, via the user interface 114. In some embodiments, whether the change of the 2D color map over time is predetermined or dynamic may be determined by the user via the user interface 114. In some embodiments, the technique for dynamically changing the 2D color map may be selected by the user via the user interface 114.

In at least one embodiment, the probe 104 may receive ultrasound echo signals from a region of interest (ROI) to generate a sequence including a plurality of temporally spaced images of the ROI. Processor 140 may calculate a parameter (e.g., TOA) for each of the plurality of temporally spaced images, and generate a color map for each of the plurality of temporally spaced images. The color map may associate the calculated parameter of a corresponding image of the plurality of temporally spaced images to a value of a color hue, a brightness, or a combination thereof. A range of the value may be based, at least in part, on a location of the corresponding image within the sequence. The processor 140 may generate a plurality of parametric images based on the color map for each of the plurality of temporally spaced images.

A graphics processor 142 may generate graphic overlays for display with the images. These graphic overlays can contain, e.g., standard identifying information such as patient name, date and time of the image, imaging parameters, and the like. For these purposes the graphics processor may be configured to receive input from the user interface 114, such as a typed patient name or other annotations. The user interface 114 can also be coupled to the multiplanar reformatter 134 for selection and control of a display of multiple multiplanar reformatted (MPR) images.

The system 100 may include a local memory 144. The local memory 144 may be implemented as any suitable non-transitory computer readable medium (e.g., flash drive, disk drive). The local memory 144 may store data generated by the system 100 including B-mode images, parametric image sequences, executable instructions, inputs provided by a user via the user interface 114, or any other information necessary for the operation of the system 100.

As mentioned previously system 100 includes the user interface 114. The user interface 114 may include the display 118 and the control panel 116. The display 118 may include a display device implemented using a variety of known display technologies, such as LCD, LED, OLED, or plasma display technology. In some examples, the display 118 may comprise multiple displays. The control panel 116 may be configured to receive user inputs (e.g., exam type, format of color map). The control panel 116 may include one or more hard controls (e.g., buttons, knobs, dials, encoders, mouse, trackball or others). In some examples, the control panel 116 may additionally or alternatively include soft controls (e.g., GUI control elements or simply, GUI controls) provided on a touch sensitive display. In some examples, the display 118 may be a touch sensitive display that includes one or more soft controls of the control panel 116.

In some examples, various components shown in FIG. 1 may be combined. For instance, the image processor 140 and the graphics processor 142 may be implemented as a single processor. In another example, the scan converter 132 and the multiplanar reformatter 134 may be implemented as a single processor. In some examples, various components shown in FIG. 1 may be implemented as separate components. For example, signal processor 122 may be implemented as separate signal processors for each imaging mode (e.g., B-mode, Doppler). In some examples, one or more of the various processors shown in FIG. 1 may be implemented by general purpose processors and/or microprocessors configured to perform the specified tasks. In some examples, one or more of the various processors may be implemented as application specific circuits. In some examples, one or more of the various processors (e.g., the image processor 140) may be implemented with one or more graphical processing units (GPU).

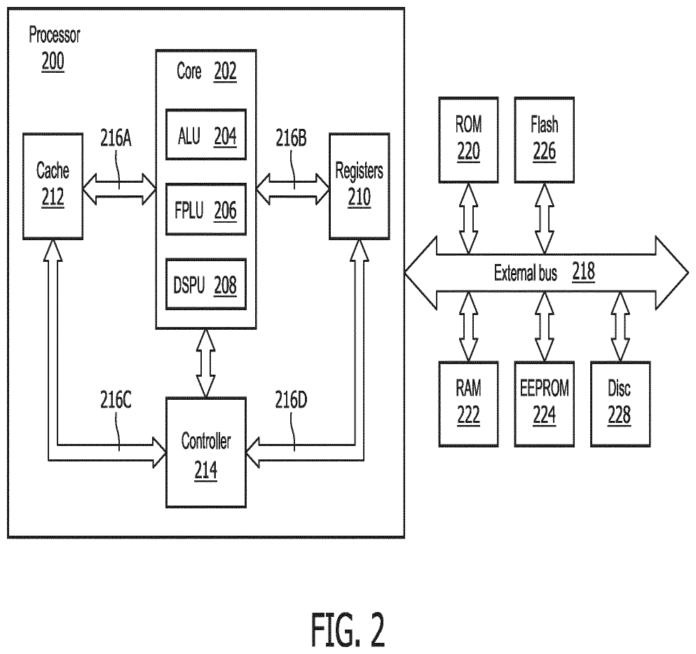

FIG. 2 is a block diagram illustrating an example processor 200 according to principles of the present disclosure. The processor 200 may be used to implement one or more processors described herein, for example, the image processor 140 shown in FIG. 1. The processor 200 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

The processor 200 may include one or more cores 202. The core 202 may include one or more arithmetic logic units (ALU) 204. In some examples, the core 202 may include a floating point logic unit (FPLU) 206 and/or a digital signal processing unit (DSPU) 208 in addition to or instead of the ALU 204.

The processor 200 may include one or more registers 210 communicatively coupled to the core 202. The registers 210 may be implemented using dedicated logic gate circuits (e.g., flip-flops) and/or any memory technology. In some examples the registers 210 may be implemented using static memory. The register may provide data, instructions and addresses to the core 202.

In some examples, processor 200 may include one or more levels of cache memory 212 communicatively coupled to the core 202. The cache memory 212 may provide computer-readable instructions to the core 202 for execution. The cache memory 212 may provide data for processing by the core 202. In some examples, the computer-readable instructions may have been provided to the cache memory 212 by a local memory, for example, local memory attached to an external bus 218. The cache memory 212 may be implemented with any suitable cache memory type, for example, metal-oxide semiconductor (MOS) memory such as static random access memory (SRAM), dynamic random access memory (DRAM), and/or any other suitable memory technology.

The processor 200 may include a controller 214, which may control input to the processor 200 from other processors and/or components included in a system (e.g., the control panel 116 and scan converter 132 shown in FIG. 1) and/or outputs from the processor 200 to other processors and/or components included in the system (e.g., the display 118 and volume renderer 136 shown in FIG. 1). The controller 214 may control the data paths in the ALU 204, the FPLU 206 and/or the DSPU 208. The controller 214 may be implemented as one or more state machines, data paths and/or dedicated control logic. The gates of the controller 214 may be implemented as standalone gates, FPGA, ASIC or any other suitable technology.

The registers 210 and the cache memory 212 may communicate with the controller 214 and the core 202 via internal connections 216A, 216B, 216C and 216D. Internal connections may implemented as a bus, multiplexor, crossbar switch, and/or any other suitable connection technology.

Inputs and outputs for the processor 200 may be provided via the external bus 218, which may include one or more conductive lines. The external bus 218 may be communicatively coupled to one or more components of processor 200, for example the controller 214, the cache memory 212, and/or the register 210. The external bus 218 may be coupled to one or more components of the system, such as the display 118 and the control panel 116 mentioned previously.

The external bus 218 may be coupled to one or more external memories. The external memories may include a read only memory (ROM) 220. The ROM 220 may be a masked ROM, an electronically programmable read only memory (EPROM) or any other suitable technology. The external memory may include a random access memory (RAM) 222. The RAM 222 may be a static RAM, battery backed up static RAM, dynamic RAM (DRAM) or any other suitable technology. The external memory may include an electrically erasable programmable read only memory (EEPROM) 224. The external memory may include a flash memory 226. The external memory may include a magnetic storage device such as a disc 228. In some examples, the external memories may be included in a system, such as ultrasound imaging system 100 shown in FIG. 1, for example the local memory 144.

As described with reference to FIG. 1, according to examples of the present disclosure, a 2D color map may be dynamic rather than fixed (e.g., predetermined, static). In some examples, a color hue value and/or brightness value range assigned to intensity values of the echo signals may dynamically change with image number of a sequence. For a temporal sequence of images, the color map may dynamically change for individual images of the sequence (e.g., the color map for an image may be based on a location of the image within the sequence). Thus, the color map changes over time. In some examples, the color hue and/or brightness distribution of the color map is progressive (e.g., linear) with time (e.g., number of images in the sequence). In some examples, as will be described in detail herein, the color hue and/or brightness distribution of the color map is adapted based, at least in part, on input image properties.

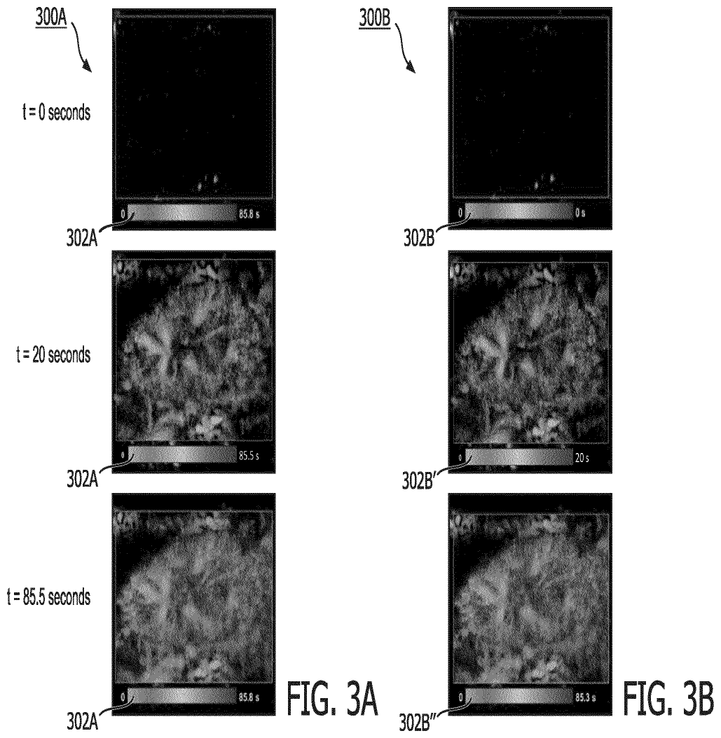

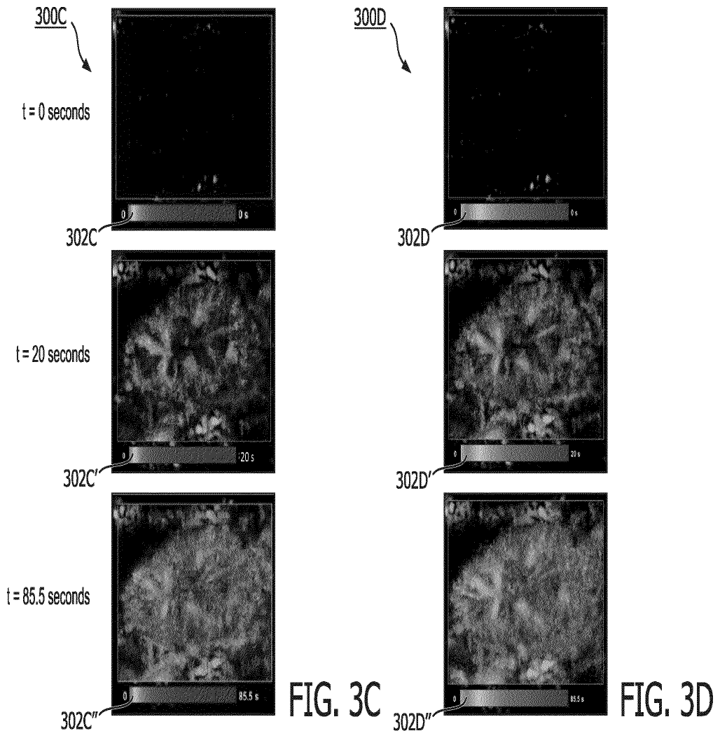

FIGS. 3A, 3B, 3C, and 3D are example TOA parametric images of sequences of a kidney generated by various CEUS techniques, including techniques according to examples of the present disclosure. In some examples, the kidney may be included in an ROI. In some examples, the entire image may be the ROI. The TOA parametric images of sequence 300A were generated with a fixed color mapping. The TOA parametric images of sequences 300B, 300C, and 300D were generated with dynamic color mappings according to embodiments of the present disclosure. The TOA parametric images of 300B were generated with a progressive dynamic color mapping technique. The TOA parametric images of sequences 300C and 300D were generated with adaptive dynamic color mapping techniques. All images of sequences 300A, 300B, 300C, and 300D were generated at time points t=0, 20 and 85.5 seconds from the same ultrasound echo signals acquired at successive time points in a time sequence between an initial time point t=0 seconds and a final time point 85.5 seconds. The TOA of a point in the ROI can be calculated as a time point when ultrasound echo signals are received from the point in the ROI. In the TOA parametric image sequences 300A, 300B, 300C, and 300D, the TOA between the initial time point t=0 seconds and the time point t at which the TOA parametric images of sequences 300A, 300B, 300C, and 300D are generated is color-coded according to the fixed color mapping, the progressive dynamic color mapping, and the adaptive dynamic color mapping, respectively.

In the TOA parametric image sequence 300A generated with a fixed color mapping, the entire color hue range between 0° and 360° (e.g., color hue numbers 0°, 120°, and 240° correspond to red, green, and blue, respectively) is assigned to the entire time range of the time sequence between the initial time point t=0 and the final time point t=85.5 seconds, as shown in the color bar 302A, irrespective of a time point at which the TOA parametric image 300A is generated. The color hue variation within the entire color hue range is linear as a function of time in the entire time range of the time sequence (e.g., 0° at t=0, 120° at t=⅓×85.5 seconds, 240° at t=⅔×85.5 seconds, 360° at t=85.5 seconds). In this example, since most of the UCA arrives (thus ultrasound echo signals from the UCA are received) in the first 10 seconds, the TOA parametric image 300A is dominated by red with no visible yellow, green, or blue. Thus, contrast of the TOA image among different pixels is low.

In the TOA parametric image sequence 300B generated with a progressive dynamic color mapping, assignment of the color hue range of 0° to 360° to a time range is dynamically changed, as shown in the color bars 302B, 302B′, and 302B″. In some examples, each image (e.g., time) within the sequence, color coding of the color map is re-computed based on the current time range of the sequence. Specifically, the color hue range of 0° to 360° is assigned to a time range of between the initial time point t=0 and the time point at which the TOA parametric image 300B is generated. For example, in the TOA parametric image 300B generated at the initial time point t=0 seconds, the entire color range is assigned to the initial time point t=0 seconds. In the TOA parametric image 300B generated at a time point t=20 seconds, the color hue range of 0° to 360° is assigned to a time range of between the initial time point t=0 seconds and the time point t=20 seconds. In the TOA parametric image 300B generated at the final time point t=85.5 seconds, the color hue range of 0° to 360° is assigned to a time range of between the initial time point t=0 seconds and the final time point t=85.5 seconds. The color hue variation within the entire color hue range is progressive with time, e.g., linear as a function of time in the dynamically changed time range (e.g., in the TOA parametric image 300B generated at the time point t=20 seconds, 0° at t=0, 120° at t=⅓×20 seconds, 240° at t=⅔×20 seconds, 360° at t=20 seconds). The visibility in the TOA image 300B generated at the time point t=20 seconds is improved, showing yellow and green pixels, as compared to the TOA image 300A at the time point t=20 seconds. However, in some instances, as shown by the image at time t=85.5 seconds, as sequence 300B becomes longer, it may suffer from similar reduced contrast issues of sequence 300A.

The progressive dynamic color mapping represents an improvement over the fixed color mapping in terms of allowing human observers to appreciate slight differences in values of the parameter to be color coded, but in some cases this improvement may not be sufficient. Adaptive dynamic color mapping, as shown in image sequences 300C and 300D, may resolve some of the issues of the progressive dynamic color mapping shown in image sequence 300B. In adaptive dynamic color mapping, the color map for an image may be generated adaptively (i.e., in a data-driven manner) based, at least in part, on at least a portion of the images of the image sequence. In some examples, the color hue and/or brightness variation may not be linear as a function of time as with the technique described with reference to sequence 300B.

In the TOA parametric images 300C and 300D generated with an adaptive dynamic color mapping, assignment of the color hue range of 0° to 360° to time is dynamically changed, in the same way as the dynamical color mapping, as shown in the color bars 302C, 302C′, and 302C″ and in the color bars 302D, 302D′, and 302D″. The color hue variation within the entire color hue range is also adaptively changed based on ultrasound echo signals acquired at successive time points between the initial time point t=0 seconds and the time point at which the TOA parametric image 300C or 300D is generated or all of the ultrasound echo signals acquired in the time sequence between the initial time point t=0 seconds and the final time point t=85.5 seconds. The adaptive dynamic color mapping is based on an occupation ratio of a number of pixels for which ultrasound echo signals have been received for the TOA parametric image 300C. The adaptive dynamic color mapping is based on a change rate of an averaged TOA value for the TOA parametric image 300D. These two techniques shown in 300C and 300D are described in more detail further below. The visibility in the TOA images 300C and 300D is improved as compared to the TOA image 300A at time points t=20 seconds and 85.5 seconds.

In the adaptive dynamic color mapping, the color hue variation is adaptively changed based on the actual values per frame of the parameter that are to be color coded, rather than merely linearly based on time. Examples of values that may be used to generate the adaptive dynamic color mapping include, but are not limited to, change rate of an average TOA value, occupation ratio, TOA percentile value, or a combination thereof.

Change rate of an averaged TOA value: The color hue distribution is dynamically changed such that a change rate of the color hue distribution is proportional to a temporal change rate of a spatially averaged TOA value. The spatially averaged TOA value at a time point t can be calculated by averaging TOA values over points in the ROI at the time point t. The change rate of the spatially averaged TOA can be calculated by taking the first time-derivative of the spatially averaged TOA. Thus, a fast changing spatially averaged TOA value leads to a fast changing color hue variation distribution. The change rate of the spatially averaged TOA value is calculated based on the ultrasound echo signals acquired between the initial time point t=0 seconds and the time point at which the TOA parametric image 300D is generated.

Occupation ratio: The color due distribution is dynamically changed such that the color due distribution is proportional to an occupation ratio of a number of pixels for which ultrasound echo signals have been received that are determined to include UCA (e.g., contrast) by the time point t at which the TOA parametric image 300C is generated to the total number of pixels in the TOA parametric image 300C. In some examples, a pixel may be determined to include the UCA based on a comparison of an intensity of the ultrasound signals received to a threshold value. For example, if the intensity of the ultrasound signals is equal to or above a threshold value, the pixel may be determined to include the UCA, and if the intensity of the ultrasound signals is below the threshold value, the pixel may be determined to not include the UCA. However, other techniques for determining which pixels include contrast may be used in other examples (e.g., analysis of Doppler data). The occupation ratio is calculated based on the ultrasound echo signals acquired between the initial time point t=0 seconds and the time point at which the TOA parametric image 300C is generated. This may be provided by Equation 1:

$$Occupation Ratio ( time ) = \qquad ( Equation 1 )$$

$$\frac{Number of pixels defined as contrast ( time )}{Total number of pixels in image} \times 100 \%$$

TOA percentile value: The color hue distribution is dynamically changed based on a percentile value of the TOA within the entire sequence. A TOA percentile value is calculated based on all of ultrasound echo signals acquired at successive time points in the time sequence. For example, 5% percentile is assigned to the minimum value (0°) of the color hue range and 95% percentile is assigned to the maximum (360°) of the color hue range.

For all adaptive dynamic color mapping techniques, the color mapping range dynamically changes with time based on the actual parameter values of each UCA frame. For each given image within the sequence, the color map is re-computed based on the data in the current image of the sequence (e.g., point in time of the sequence for a temporal sequence of images) and the chosen algorithm (e.g. change rate, occupation rate, percentile).

Furthermore, while the examples provided in FIGS. 3A-3D refer to parametric images for TOA, dynamic color mapping techniques may be used for other parameters such as a time-to-peak, a wash-out rate, a concentration, a flow rate, or a perfusion rate or a combination thereof of an ultrasound contrast agent administered in the ROI in CEUS images.

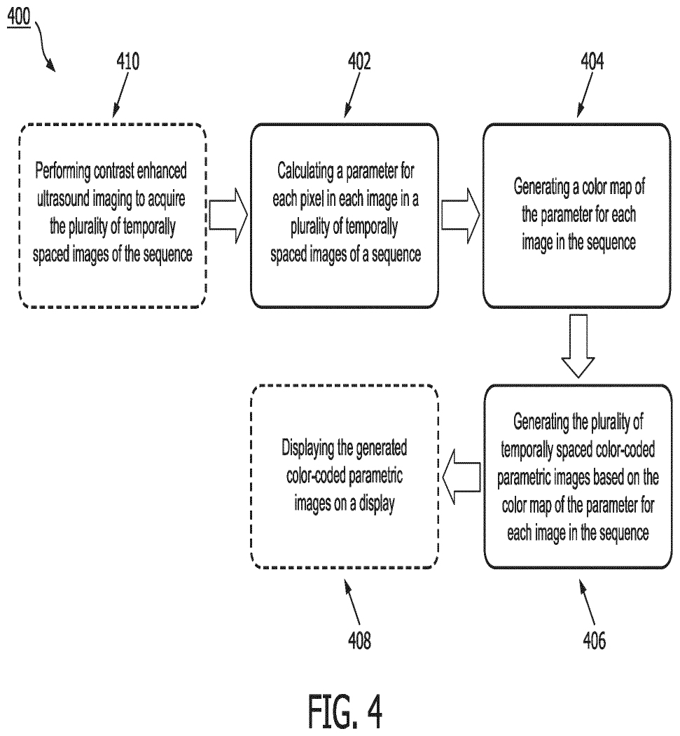

FIG. 4 is a flow chart of a method 400 of forming a color-coded parametric image of a region of interest (ROI), according to an embodiment of the present disclosure. In some embodiments, some or all of the method 400 are performed by the imaging system 100 shown in FIG. 1. In the embodiments described herein, a color-coded TOA parametric image of the ROI is used as an example. However, the method 400 may be beneficially utilized to map other time parameters related to the ROI, such as a time-of-arrival, a time-to-peak, a wash-out rate, a concentration, a flow rate, or a perfusion rate of an ultrasound contrast agent administered in the ROI in CEUS images.

At block 402 “calculating a parameter for each pixel in each image in a plurality of temporally spaced images of a sequence” may be performed. In some examples, block 402 may be performed by one or more processors, such as processor 200 and/or processor 140. In some examples, the images may be ultrasound images acquired by an ultrasound imaging system, such as imaging system 100. In some examples, the parameter may include a time of arrival (TOA), a time-to-peak, a wash-in rate, a wash-out rate, a concentration, a flow rate, a perfusion rate, or a combination thereof.

At block 404, “generating a color map of the parameter for each image in the sequence” may be performed. The color map may include values of a color, a brightness, or a combination thereof to be assigned to each pixel of the image, based at least in part on a range of the calculated parameter to be color coded in some examples. In some examples, a range of the value is based, at least in part, on a temporal location of the corresponding image within the sequence. In some examples, the color map may be a 2D color map. In some examples, block 404 may be performed by one or more processors such as processor 200 and/or processor 140. In a temporal sequence of images (e.g., a cineloop), each image of the sequence may have been acquired at a different point in time and thus may represent a particular time point in the sequence. For example, a first image of a sequence may represent an initial time t=0 and a final image of the sequence may represent a final time t=N, where N may be some number of units of time (e.g., seconds, minutes).

The color map may be generated by various techniques, such as those described herein, including those with reference to FIGS. 3A-3D. In some examples, for generating the color map, a variation of the value within the range of the value is adaptively changed based on a change rate of a spatially averaged value of the parameter. In some examples, a variation is adaptively changed based on a percentile of the parameter. In some examples, a variation of the value within the range of the value is linear as a function of time. In some examples, a variation of the value within the range is adaptively changed based on an occupation ratio. In some examples, the occupation ratio is calculated based, at least in part, on a ratio of pixels including a contrast agent and a total number of pixels in an image of the plurality of temporally spaced images.

At block 406, “generating the plurality of temporally spaced color-coded parametric images based on the color map of the parameter for each image in the sequence” may be performed. In some examples, block 406 may be performed by one or more processors, such as processor 200 and/or processor 140.

Optionally, method 400 may further include displaying the generated color-coded parametric images on a display, such as display 118, as indicated by block 408. In some examples, the parametric images may be displayed overlaid on the images of the sequence.

Optionally, prior to block 402, method 400 may further include “performing contrast enhanced ultrasound imaging to acquire the plurality of temporally spaced images of the sequence” as indicated by block 410. In some examples, the contrast enhanced ultrasound imaging may be performed by an ultrasound imaging system, such as system 100. In some examples, an ultrasound probe, such as probe 104 may be used to transmit ultrasound signals and receive resulting echo signals to generate the images.

An example of a method according to the disclosure relating to dynamic color mapping for a time sequence of images for time-of-arrival (TOA) of an ultrasound contrast agent (UCA) is provided. However, this is merely for illustration, and other parameters (e.g., time-to-peak, wash-out) may be used in other examples.

The TOA for the UCA is calculated by a processor, for example, the processor 200 and/or processor 140, for each of temporally spaced images of a sequence, based at least in part on ultrasound echo signals from a ROI in a subject received at successive time points in a time sequence to generate images at the time points. The ultrasound echo signals are received using an ultrasound probe, for example, the ultrasound probe 104. In the sequence, an initial time point t=0 may correspond to the time when UCA is administered in a subject, and ultrasound echo signals from the UCA are acquired at successive time points between the initial time point t=0 and a final time point of the time sequence. In some embodiments, a sequence of ultrasound images is generated from the acquired ultrasound echo signals. The sequence of ultrasound images may be generated, at least in part, by a signal processor, a B-mode processor, a Doppler processor, a scan converter, and/or the image processor, for example, the image processor 140. A TOA of the UCA at a point in the ROI is calculated as a point when ultrasound echo signals are received from the point in the ROI.

A color map may associate a TOA to a color hue value and/or brightness value for each of the temporally spaced images of the sequence at a time point t between the initial time and the final time in the time sequence. The color map may be a progressive dynamic color map or an adaptive dynamic color map.

To generate the progressive dynamic color map at a time point t, the color hue range of 0° to 360° is assigned to a time range of between the initial time point t=0 seconds and the time point 1. The color hue variation within the entire color hue range is linear as a function of time in the dynamically changed time range.

To generate the adaptive dynamic color mapping, the color hue range of 0° to 360° is assigned to a time range of between the initial time point t=0 and the time point t. The color hue variation within the entire color hue range is also adaptively changed based on ultrasound echo signals acquired at successive time points between the initial time point t=0 seconds and the time point t or all of the ultrasound echo signals acquired between the initial time point t=0 seconds and the final time point. The color hue variation is adaptively changed based on a change rate of a spatially averaged TOA value, an occupation ratio, or a TOA percentile value, as described above.

Temporally spaced images of the sequence are generated, based on the color map generated for each of the temporally spaced images of the sequence. A color hue of each pixel in a TOA parametric image represents a TOA of the UCA at a corresponding point in the ROI. A TOA parametric image at a time point t shows TOA values that are between the initial time point t=0 seconds and the time point t. The generated TOA parametric images are displayed by a display, such as the display 118.

Optionally, in some embodiments, the method further includes receiving a user input, via a user interface, such as the user interface 114. In some embodiments, the user input may be used to select between the fixed color mapping, the progressive dynamic color mapping, and the adaptive dynamic mapping. The user input may further be used to select how the color hue variation is adaptively changed (e.g., based on a change rate of a spatially averaged TOA, an occupation ratio, or a TOA percentile value).

In the examples described herein, a color hue value is used as an example of a color value that is dynamically changed. However, the method 400 may be beneficially utilized to dynamically change other color values, such as a brightness value that ranges between 0% and 100% and a saturation value that ranges between 0% and 100%.

As described herein, a color map, such as a 2D color map, that may vary in time may be used for forming a time sequence of parametric images. In some examples, a color map is dynamically adjusted based on a time point within the time sequence. In some examples, a color map is progressively adjusted based on a time point within the time sequence and/or adaptively adjusted based on a time parameter of interested at all or some of the time points within the time sequence. In some applications, parametric images formed according to principles of the present disclosure may allow for larger variation in color hue and thus providing increased visual distinguishability from one pixel to another.

In various examples where components, systems and/or methods are implemented using a programmable device, such as a computer-based system or programmable logic, it should be appreciated that the above-described systems and methods can be implemented using any of various known or later developed programming languages, such as “C”, “C++”, “Python”, and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software, and/or firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instructions to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, examples or processes described herein may be combined with one or more other examples, examples and/or processes or be separated and/or performed amongst separate devices or device portions in accordance with the present systems, devices and methods.

Finally, the above-discussion is intended to be merely illustrative of the present systems and methods and should not be construed as limiting the appended claims to any particular example or group of examples. Thus, while the present system has been described in particular detail with reference to exemplary examples, it should also be appreciated that numerous modifications and alternative examples may be devised by those having ordinary skill in the art without departing from the broader and intended spirit and scope of the present systems and methods as set forth in the claims that follow. Accordingly, the specification and drawings are to be regarded in an illustrative manner and are not intended to limit the scope of the appended claims.

1. An ultrasound imaging system comprising:
an input configured to receive ultrasound echo signals from a region of interest (ROI) to generate a sequence comprising a plurality of temporally spaced color-coded parametric images of the ROI; and
a processor configured to:
calculate a parameter for each pixel in each image in the sequence;
generate a color map of the parameter for each image in the sequence, wherein the color map includes values of a color hue, a brightness, or a combination thereof to be assigned to each pixel of the image, based at least in part on a range of the calculated parameter to be color coded, wherein a range of the values of the color map is determined based, at least in part, on a temporal location of the corresponding image within the sequence; and
generate the plurality of temporally spaced color-coded parametric images based on the color map of the parameter for each image in the sequence.

2. The ultrasound imaging system of claim 1, wherein the color map of the parameter varies linearly as a function of time.

3. The ultrasound imaging system of claim 1, wherein the color map of the parameter varies adaptively based on a change rate of a spatially averaged value of the parameter.

4. The ultrasound imaging system of claim 1, wherein the color map of the parameter varies adaptively based on an occupation ratio.

5. The ultrasound imaging system of claim 4, wherein the occupation ratio is calculated based, at least in part, on a ratio of pixels including a contrast agent and a total number of pixels in an image of the plurality of temporally spaced images.

6. The ultrasound imaging system of claim 1, wherein the color map of the parameter varies adaptively based on a percentile of the parameter.

7. The ultrasound imaging system of claim 1, further comprising a display configured to display the plurality of temporally spaced color-coded parametric images.

8. The ultrasound imaging system of claim 1, wherein the input comprises an ultrasound probe.

9. A method of forming a color-coded parametric image sequence, comprising:
calculating a parameter for each pixel in each image in the sequence;
generating a color map of the parameter for each image in the sequence, wherein the color map includes values of a color hue, a brightness, or a combination thereof to be assigned to each pixel of the image, based at least in part on a range of the calculated parameter to be color coded, wherein a range of the values of the color map is determined based, at least in part, on a temporal location of the corresponding image within the sequence; and
generating the plurality of temporally spaced color-coded parametric images based on the color map of the parameter for each image in the sequence.

10. The method of claim 9, wherein the parameter comprises a time of arrival (TOA), a time-to-peak, a wash-in rate, a wash-out rate, a concentration, a flow rate, a perfusion rate, or a combination thereof.

11. The method of claim 9, further comprising performing contrast enhanced ultrasound imaging to acquire the plurality of temporally spaced images of the sequence.

12. The method of claim 9, wherein the color map of the parameter varies adaptively based on an occupation ratio.

13. The method of claim 12, wherein the occupation ratio is calculated based, at least in part, on a ratio of pixels including a contrast agent and a total number of pixels in an image of the plurality of temporally spaced images.

14. The method of claim 9, wherein the color map of the parameter varies linearly as a function of time.

15. The method of claim 9, wherein the color map of the parameter varies adaptively based on a change rate of a spatially averaged value of the parameter.

16. The method of claim 9, wherein the color map of the parameter varies adaptively based on a percentile of the parameter.

17. A non-transitory computer readable medium including instructions that when executed cause an ultrasound imaging system to:
calculate a parameter for each pixel in each image in the sequence;
generate a color map of the parameter for each image in the sequence, wherein the color map includes values of a color hue, a brightness, or a combination thereof to be assigned to each pixel of the image, based at least in part on a range of the calculated parameter to be color coded, wherein a range of the values of the color map is determined based, at least in part, on a temporal location of the corresponding image within the sequence; and
generate the plurality of temporally spaced color-coded parametric images based on the color map of the parameter for each image in the sequence.

18. The non-transitory computer readable medium of claim 17, further comprising instructions that when executed cause the ultrasound imaging system to:
select the parameter to calculate from a plurality of parameters based, at least in part, on a user input received from a user interface.

19. The non-transitory computer readable medium of claim 18, wherein the plurality of parameters comprises a time of arrival (TOA), a time-to-peak, a wash-in rate, a wash-out rate, a concentration, a flow rate, a perfusion rate, or a combination thereof.

20. The non-transitory computer readable medium of claim 17, wherein the color map of the parameter varies linearly as a function of time or varies adaptively based on an occupation ratio or a change rate of a spatially averaged value of the parameter.

21. The non-transitory computer readable medium of claim 17, further comprising instructions that when executed cause the ultrasound imaging system to:
display the plurality of temporally spaced color-coded parametric images on a display.
